(19) United States
(12) Patent Application Publication
(10) Pub. No.: US 2010/0204542 A1
(43) Pub. Date: Aug. 12, 2010

(54) MALE MEMBER ACCESSORY

(76) Inventor: Brian J. Hodge, New South Wales (AU)

Correspondence Address: TRASKBRITT, P.C., P.O. BOX 2550, SALT LAKE CITY, UT, 84110, US

(21) Appl. No.: 12/448,786

(22) PCT Filed: Jan. 9, 2008
(86) PCT No.: PCT/AU2008/000012

§ 371 Date: Sep. 18, 2009

(30) Foreign Application Priority Data
Jan. 9, 2007 (AU) 2007900049

(51) Int. Cl. A61F 5/00; A61H 1/00; A61F 6/04

(52) U.S. Cl. 600/39; 601/46; 128/844

(57) ABSTRACT

A penis accessory (2) for enlarging the effective male sex apparatus, the accessory (2) mountable to the penis (14) of a male for augmenting the penile size and/or penile shape and/or enhancing sexual performance of the male wearer, the accessory (2) including (i) a first part (10) having a head portion (20) for fitting over the glans penis and a body portion (22) for at least partially covering the penile shaft (14), the body portion (22) optionally having dependent fingers (24) having thick walls that augment the size of the wearer's penis; and a second part (12) acting as retaining means to retain the first part (10) on the shaft (14).

### FIELD OF INVENTION

This invention relates to an accessory for augmenting the site, shape and/or performance of the male member or sex organ, the penis. More particularly, this invention relates to a penis accessory having multiple parts.

### BACKGROUND ART

The following references to and descriptions of prior proposals or products are not intended to be, and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

The augmentation of the male human sex organ for improved performance and satisfaction to the owner and his partner has been the subject of considerable activity and represents a significant, if specialised, commercially thriving industry. Although activity in this area has been intense, efforts to provide a means of enhancement of penile performance without medication have been less than successful.

Condoms have been available for the reduction of the incidence of sexually transmitted diseases, but such devices are not primarily for augmentation of the penis. Indeed, in seeking to enhance penile size, such devices have tended to lead to diminution of sexual pleasure, due to thickening of the walls of the device, resulting in reduced sensation to the male and disappointment to the partner.

The male sexual organ to which the present invention relates consists of the penis. The penis includes a shaft member that extends out of the body and terminates in a bulbous head member called the glans. Internally, the penis has two dorsal and one ventral spongy tubes or tissue columns that can become engorged with blood to cause an erection. One of the spongy tubes (corpus spongiosum) contains the urethra and extends along the ventral side in the penis. The urethra terminates at the urethral opening or meatus. A sensitive part of the penis is the area on the ventral side in the region between the glans and the underside of the shaft called the frenum which is where the foreskin in uncircumcised males is attached. A visible seam between the lateral halves of the penis is the raphe that extends centrally along the ventral or underside of the penis running from the meatus, past the scrotum to the perineum (i.e.

An object of the present invention is to ameliorate the aforementioned disadvantages of the prior art or to at least provide a useful alternative thereto.

### STATEMENT OF INVENTION

Accordingly, in one aspect of the invention there is provided:
a penis accessory mountable to the penis of a male for augmenting the penile size and/or penile shape and/or enhancing sexual performance of the male wearer, the accessory including:
   a first part having a head portion for fitting over the glans and a body portion for at least partially covering the penile shaft;
   a second part to retain the first part on the shaft.

The second part may be separate from, integrally formed with or attached to the first part, or may form part of the first part. The augmentation achieved by the accessory may be to increase the effective size of the penis in that the whole apparatus including the penis and the accessory is larger, thicker and/or longer than the penis on its own.

The size of the penis may be augmented in length by the application of the head portion that extends longitudinally beyond the glans and meatus. The thickness of the penis may be augmented by the body portion that extends, at least in part along the shaft and effectively widens the diameter of the male apparatus that enters a partner's canal during intercourse.

The shape of the penis may be enhanced by providing an exaggerated glans member and thickened shaft member that can provide increased stimulation to the partner during sexual play and intercourse.

The male wearer's sexual performance may be enhanced by the first part by causing increased stimulation of the meatus, the frenulum and/or the shaft.

The penis accessory may be a unitary item in which the first and second parts are joined. For example, the first part may include a loosely or firmly fitting sleeve that fits over the shaft. The sleeve may be of a generally cylindrical construction. The sleeve may comprise a wall or walls of uniform thickness. Preferably, however, the body portion includes portions of variable thickness. Preferably, the variable portions may be rib or ridge structures with complementary groove features. The variable portions may be longitudinally, laterally or diagonally (such as spirally or helically) arranged. The variable portions may be knobs or nodules that are regularly arranged in grid or other regular patterns or randomly arranged.

Alternatively, the accessory may comprise separate or separable first and second parts. The accessory may comprise three or more parts, for example a shaft part, a head part and a retaining part.

The male wearer is preferably a healthy male human. However, the wearer may be a person suffering from an erectile dysfunction condition which can be improved by increased stimulation to the general shaft region and the glans to facilitate engorgement of the dorsal spongy tubes (corpora cavernosa) and the corpus spongiosum. In any case, even a healthy male may derive improved stimulation from the penis accessory whereby to achieve improved and longer lasting sexual performance.

The first part preferably effects augmentation of penis shape. For example, it may provide extra length and/or thickness to the penis. It may provide a variation in shape, particularly changes in contour such as by utilising ribs or lumps or other variations not normally found on a penis.

The head portion may comprise a relatively firm or solid tip that is smoothly contoured so as not to cause discomfort to the wearer's partner. The head may be provided in different shapes, as desired. The head portion may include a vibrator to enhance stimulation. The head portion may be soft and bulbous and of similar consistency to a natural glans, optionally just larger. The head portion preferably includes a hollow reservoir for retaining seminal fluid in a manner similar to a condom. The head portion is preferably hollow and sufficiently malleable or flexible to accommodate a range of glans shapes and sizes. Alternatively, the head portion is provided in a range of dimensions to accommodate different sized appendages.

The head portion may comprise a wall or walls made of thin material, such as a thin film of latex or polyurethane as found in condoms. The head portion may include a thickened wall of resilient spongy material formed from natural or synthetic rubber as an internal layer, noting that the external filmic surface is preferably made of a sperm and disease impervious barrier such as latex, polyurethane or silicone, preferably medical grade silicone. Preferably, the head portion is non-porous and impervious to liquids. The hollow head portion may include a lubricant, spermicidal and/or other biocidal liquid, substance and/or layer whereby to enhance stimulation and/or to reduce the risk of disease transmission. The head portion may include internal contour or surface feature variations, such as lumps and/or ribs for increased stimulation to the wearer.

The body portion preferably includes one or more longitudinal slits or expansion lines defining one or more longitudinal fingers or strips of material depending from the head portion. The one or more slits may be in the form of at least one gap between the fingers of the opposed edges of a finger and may enable the finger or the fingers to expand or spread to fit over the glans on application to the shaft of the male's member and/or to accommodate different sized male members. The one or more fingers may comprise thickened lengths of material having contoured features such as ribs, ridges, knobs or protrusions and the like. In one embodiment, these features may be applied facing inward to improve pleasure for the wearer. Alternatively, the features may be designed to face outwardly for the benefit of his partner.

The fingers may terminate at the ends extending towards the shaft base in “free” ends. The fingers may be joined at their “free” ends by a ring or circular ribbon to maintain relatively even spacing between the fingers. Alternatively, the respective free ends of the fingers are unattached to their adjacent fingers.

The second part may be a sheath that can be usefully slid or rolled over the first part covering the glans and onto the shaft. The second part has preferably a cylindrical shape. It may be transferred to the wearer in a rolled configuration and unrolled from the glans to the shaft base in the same manner as a condom. The second part may include a head cover adapted to cover the head portion. The second part may be a condom.

The second part is preferably a retaining means to retain the first part in place. This is particularly useful where the fingers are not joined at their free ends and the first part does not seal the male member against infectious contact or transfer.

The first part may have a soft covering over a harder, inner core layer. The soft covering may line the inner and outer surfaces of part or all of the first part. The first part may be made of silicone, polyurethane, latex and/or another soft plastic material, but preferably medical grade silicone, noting that the properties of any material used should comply with international standards (ISO 4074-1:1996(E)). The first part may have a sticky or tacky texture and/or surface features that assist in providing grip and in retaining the first part in place. Preferably, the first and second parts are lubricated. The tip or head portion of the accessory covering part or all of the glans, whether forming part of the first and/or second parts, may be hardened for greater strength.

The first part may be used alone, but it is noted that the second part, in such circumstances will form part of the first part to ensure the first part is usefully secured in place. However, preferably the second part is a distinctive component to the first part and is provided to act as retaining means for the first part. Nevertheless, an alternative arrangement includes providing stretchable, elastic material between the fingers to perform as the second part retaining means. The bridging retaining means between the fingers may be of a similar consistency and thickness to standard condom material and may be made of latex or polyurethane, but preferably silicone, still more preferably medical grade silicone. The first part may be made from an homogenous material with variations in thickness of component parts being the primary variable to achieve different functionality of each component part, such as the fingers, bridging retaining means and the head portion. In this connection, the first and second parts may be made by an injection moulding process in a manner similar to conventional condom manufacture, although complex curves and features of variable thickness may require moulding of separate halves or a single substantially planar blank that may be joined at opposed edges to form a seam. However, in a preferred embodiment, the first part is formed from multiple layers of different types of materials, such as a soft and tacky inner layer bonded to a harder outer layer. Preferably, the first and second parts are moulded under pressure. The accessory parts may be made of silicone or a silicon based is material, preferably medical grade silicone.

The retaining means may be adapted to wrap over the first part. The retaining means may have a sticky or tacky texture and/or surface features so as to assist in providing grip and in retaining the retaining means in place. The retaining means may be provided in any suitable form. For example, the retaining means may be provided in a sheet, in a square, rectangle or other desired shape, to wrap, surround, cover and/or otherwise retain the first part. In such arrangements, the free longitudinal edges to be joined may be respectively lined with a pressure sensitive, repositionable glue of sufficient tackiness to withstand the rigours of use.

Alternatively, the retaining means may be tubular whereby to stretch over the glans for positioning on the shaft.

The retaining means may cover only the body portion, leaving the head portion free or both the body and the head portion of the first part may be covered by the retaining means. In the case where the body portion does not cover the whole of the shaft, for example where the body portion comprises longitudinally extending fingers separated by gaps, the retaining means may be intended to have direct contact with the shaft so as to assist in keeping the accessory in place.

It is preferable that the retaining means is made of flexible, stretchy material, so that it can be rolled along or wrapped around the circumference of the shaft to facilitate application and removal of the retaining means. The flexible, stretchy material may include soft latex or soft plastic, or may be made from silicone or a silicon based material, preferably medical grade silicone.

The retaining means may include physical structures to uniformly or selectively expand its effective diameter about the glans and/or the shaft. The physical structures may be in the form of lumps, protrusions, knobs, ribs, ridges extending laterally, longitudinally or diagonally, or a combination of two or more of the proceeding. The physical structures of the retaining means may be complementary to the physical structures of the body portion. Alternatively, the physical features of the retaining means may be complementary to the corresponding physical features of the body portion and/or the head portion. As an example, lumps and/or ribs may be provided under or between layers of, the first part and/or the retaining means.

The physical structures may include a network of fluid cells and/or conduits to provide cushioning and improved sensation. The physical structures may be adapted to retain air, water or lubricant, or to receive same via suitable valves and/or inlet apertures.

The retaining means may be joined to or separate from the first part. The retaining means may be, in storage, in rolled or folded form, and attached to either the top or the base of the body portion. Where the retaining means is separate from the first part, the retaining means may be separately packaged, or may be packaged together with the first part. The retaining means may be a condom.

It is preferable that a condom is used in conjunction with either the first or the second part when used alone, or both the first and second parts when used together, to assist in ensuring that the accessory remains in place and to reduce the chance of encountering seminal leakage and/or microbiological infections.

The accessory may be fitted to an inanimate object, such as a vibrator.

### BRIEF DESCRIPTION OF THE DRAWINGS

Preferred features of the present invention will now be described with particular reference to the accompanying drawings. However, it is to be understood that the features illustrated in and described with reference to the drawings are not to be construed as limiting on the scope of the invention. In the drawings:

FIG. 1a is a side elevation of a first embodiment of the accessory of the present invention;

FIG. 1b is a side elevation of a second part of the accessory shown in FIG. 1a which is turned inside out;

FIG. 1c is a side elevation of the first part according to one embodiment of the invention;

FIG. 2 is a side elevation of a second part according to another embodiment of the present invention;

FIG. 3 is a plan view of a second part according to another embodiment of the invention; and

FIG. 4 is a perspective view of yet another embodiment of the present invention.

### DETAILED DESCRIPTION OF THE DRAWINGS

It will be readily appreciated that the views in the various figures are not necessarily drawn to the same scale.

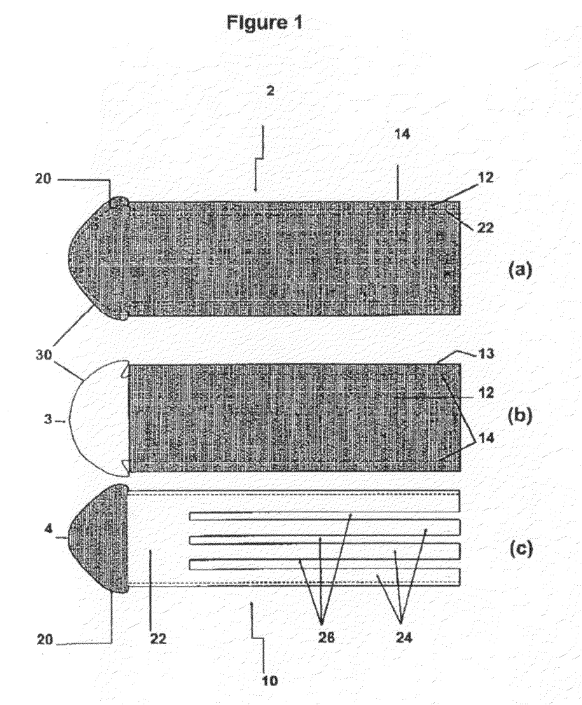

As shown in FIG. 1a, penis accessory 2 includes a first part 10 adapted to cover a penis. In this embodiment, the accessory 2 includes a head 20 which is intended to cover the glans (obscured). The first part 10 also includes a body portion extending along the shaft which is enveloped by a second part 12 in the form of a sheath, when in use. The second part 12 can also wrap over the whole first part (the head portion 20 and the body portion (obscured)). The second part 12 may be in a tubular form, whereby it is slid over the glans and secured on the shaft. Both the first part 10 and the second part 12 may be used with a condom (not shown). Accordingly, in use, the first part 10 is firstly placed on the penis and located in position, where after. the second part 12 is slid over or wrapped around the glans and/or shaft to act as a retaining means for the first part 10.

As shown in FIG. 1a, the head portion 20 is covered by the second part 12 in this embodiment.

As shown in FIG. 1b, the second part 12 may be in the form of a tube having thickened walls whereby to space the external surface 13 of the second part 12 from the shaft surface 14, whereby the diameter of the second part 12 is significantly greater than that of the shaft 14. Accordingly, the second part 12 may be used around to add thickness to the shaft 14. Alternatively, the second part 12 can be provided in a sheet form as shown in FIG. 3, wrapping the shaft 14 to increase thickness. Lumps and/or ribs, knobs, ridges, corrugations and/or grooves may be provided on the inner or outer surfaces of the second part, or between layers thereof to increase sensation of the area and for his partner.

In FIG. 1c, the first part 10 includes a head portion 20 to cover the glans (not shown) and the head portion 20 is attached to a body portion 22 by being injection moulded from a single source of material, such as latex or polyurethane, or silicone, preferably medical grade silicone. Consequently, the head portion 20 and the body portion 22 may be integrally formed under pressure by the injection moulding process. The components may alternatively be separately formed and subsequently attached by heat welding and/or joined using non-allergenic adhesive, and/or formed in a multi-step process in which multiple layers are formed.

Referring to FIGS. 1b and c, the second part 12 as shown in FIG. 1b has its inside facing outward. The second part 12 is intended to encapsulate the first part 10. To this end, the second part 12 may be aligned with the first part 10 having the respective ends 3 and 4 pointing towards each other. As the second part 12 is forced against the first part 10, the second part 12, which has its inner side out, will reverse to extend over the head portion 20 and body portion 22 of the first part 10. This results in the originally outward facing inner side of an end cover 30 covering the head portion 20 and the external surface 13 of the second part 12 covering the body portion 22.

The body portion 22 is adapted to fit over the shaft. The body portion 22 includes a plurality of downwardly depending fingers 24 constituted by one or more slits 26 between adjacent fingers 24. The material and thickness of the first part 10 may be such that the forgers 24 are able to maintain form when placed over the penis, but will generally not hold form on their own if subject to the rigours of use. This may facilitate ease of positioning of the fingers 24 when fitting the retaining means 12 thereover. The fingers 24 are preferably of sufficient thickness (for example, 1-5 mm, 1-10 mm or more) to provide variation in size of the shaft. The fingers 24 may be spread apart to accommodate a large shaft, or may be packed together or even overlapping to accommodate a smaller shaft. In this embodiment, the head 20a is provided as a solid tip made of medical grade silicone with an option of a built in vibrator. In use, the second part 12 may be applied over the first part 10 to secure the fingers 24 in position, preferably allowing the fingers 24 to be regularly spaced around the circumference of the shaft.

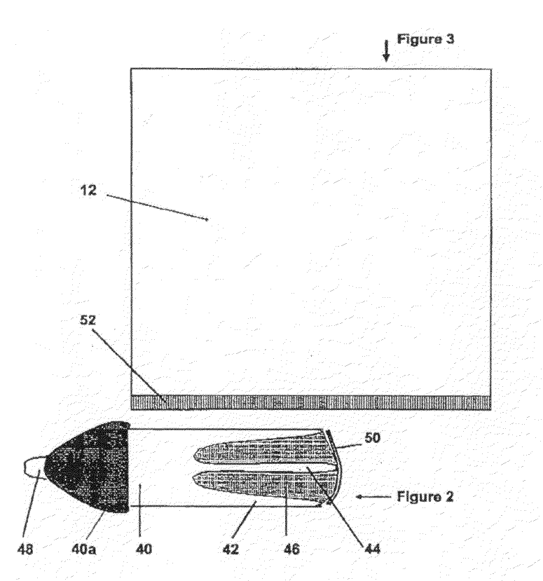

With regard to FIG. 2, there is shown another form of the first part 40 providing an overall different shape with regard to the body portion 42 to effect a different shapedresultant male member apparatus. In this form of the first part 40, fingers 44 are separated by large gaps 46, so that the resultant accessory will have pronounced longitudinal ridges corresponding to the fingers 44 when in use. The first part 40 includes a head portion 30a with a small hollow tip 48 to provide a reservoir for seminal fluid. Optionally, the fingers 44 are joined by a base ring 50 of flexible material whereby to ensure that the fingers 44 are equally spaced in use. In another variation to the first part 40, the gaps 46 may be filled with a bridging film extending in the form of webs between the fingers 44 to provide a stretchable and elastic web adapted to ensure that the first part 40 can be stretched over the penis and secured under tension. In such an embodiment, the second part retaining means is considered to be the web. The ring 50 may be in the form of a flat ribbon to minimise any tourniquet affect.

The head portion 40a may be provided internally with a lubricating film or substance and/or a biocide.

FIG. 2 also indicates that the body 42 of the first part 40 may not necessarily cover the entire shaft as this will enable the second part 12 (not shown) to have direct contact with the shaft. In such an arrangement, of course the web material at position 46 will not be present.

As shown in FIG. 3, the second part 12 may be in the form of a rectangular wrap of filmic material that has a strip of repositionable adhesive material 52 adapted to releasably join to the remainder of the second part 12 material, once the second part 12 is wrapped around the shaft and over the first part 10. Alternatively, the material of the second part 12 is self-adhering polymeric material (such as low density polyethylene), whereby not requiring an adhesive strip 52.

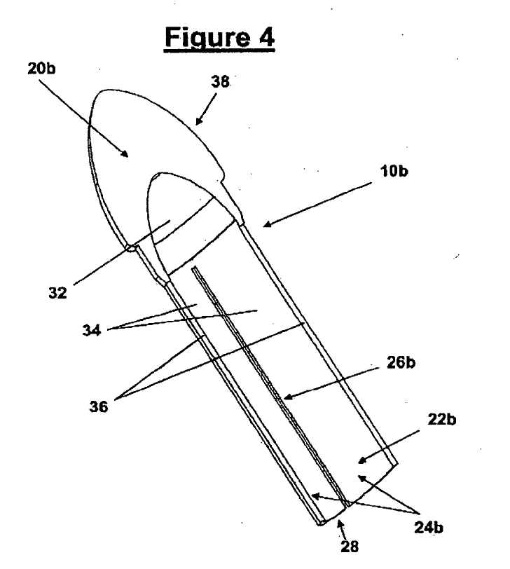

In FIG. 4 there is shown another embodiment of the invention including a first part 10 comprising a head portion 20b and a body portion 22b. The body portion 22b includes four equally spaced fingers 24b, each finger 24b separated from its adjacent pair by a longitudinal slit or gap 26b extending from near the base of the glans 30 to the base of the shaft 28. The first part 10b is internally hollow and defined by internal walls 32, 34.The fingers 24b are sufficiently thick to add to the diameter of the shaft 14 (not shown) of the wearer by a thickness indicated by the wall. 36 which may vary between 0.5-10 mm. The slit or gap 26b allows expansion to accommodate different wearer sizes.

The head portion 20b comprises a bulbous tip 38 made from relatively spongy-tissue polymeric material, such as polyurethane, optionally impregnated with a lubricant. Alternatively, the tip 38 may be made from a hard or solid material, such as a silicone, preferably medical grade silicone, optionally equipped with a small vibrator.

Through-out the specification and claims the word “comprise” and its derivatives is intended to have an inclusive rather than exclusive meaning unless the context requires otherwise.

Orientational terms used in the specification and claims such as vertical, horizontal, top, bottom, upper and lower are to be interpreted as relational and are based on the premise that the component, item, article, apparatus, device or instrument will usually be considered in a particular orientation, typically with the head portion uppermost.

It will be appreciated by those skilled in the art that many modifications and variations may be made to the methods of the invention described herein without departing from the spirit and scope of the invention.

1. A penis accessory mountable to the male sex apparatus of a male for augmenting penile size and/or penile shape and/or enhancing sexual performance of a male wearer, the accessory including:
a first part having a head portion for fitting over the glans and a body portion for at least partially covering the penile shaft; and
a second part to retain the first part on the penile shaft,
wherein said second part is separable from the first part and at least fully covers the body portion.

2. The accessory according to claim 1, wherein said body portion includes at least one finger extending longitudinally along a length of said penile shaft away from said head portion.

3. The accessory according to claim 1, wherein each finger or finger edge is separated along its length from each adjacent finger or finger edge by a gap.

4. The accessory according to claim 1, wherein the length of each finger is separated from each adjacent finger at least partially by a web.

5. The accessory according to claim 1, wherein at least one of said fingers comprises a thick wall whereby to effectively thicken the male sex apparatus comprising the penis and the accessory.

6. The accessory according to claim 1, wherein at least one of said fingers or said second part includes one or more physical structures.

7. The accessory according to claim 6, wherein said one or more physical structures include at least one feature from the group of knobs, nodules, ridges, ribs, grooves and corrugations.

8. The accessory according to claim 7, wherein said physical structures are randomly placed.

9. The accessory according to claim 7, wherein said physical structures are longitudinally, laterally or diagonally aligned, or a combination of one or more of the preceding.

10. The accessory according to claim 1, wherein said second part is a retaining means made of flexible material that can be rolled along or wrapped around the circumference of said penile shaft to facilitate application and removal of said retaining means.

11. The accessory according to claim 2, wherein at least one of said fingers is laterally aligned.

12. The accessory according to claim 2, wherein at least one of said fingers is diagonally aligned.

13. The accessory according to claim 1, wherein said second part is a retaining means that is adapted to wrap around said body portion to retain said body portion in place on said shaft.

14. The accessory according to claim 13, wherein said retaining means wraps around said head portion.

15. The accessory according to claim 1, wherein said second part extends substantially the full length of said penile shaft.

16. The accessory according to claim 1, wherein said second part is attached to the first part.

17. The accessory according to claim 1, wherein said head portion is a solid tip.

18. The accessory according to claim 1, wherein said head portion is hollow.

19. The accessory according to claim 1, wherein said head portion includes a vibrator.

20. The accessory according to claim 1, wherein said second part is a condom.
